(19) United States
(12) Patent Application Publication
(10) Pub. No.: US 2008/0249063 A1
(43) Pub. Date: Oct. 9, 2008

(54) Composition for promoting the maintenance and function of muscle-specific progenitor cells

(76) Inventors: Marvin A. Heuer, Mississauga (CA); Ken Clement, Mississauga (CA); Shan Chaudhuri, Mississauga (CA); Michele Molino, Mississauga (CA); Philip Apong, Mississauga (CA); Jason Peters, Mississauga (CA)

Correspondence Address: IOVATE HEALTH SCIENCE RESEARCH INC., 381 North Service Road West, Oakville, ON, L6M 0H4, CA

(21) Appl. No.: 11/784,098

(22) Filed: Apr. 4, 2007

(51) Int. Cl. A61K 31/195; A61K 31/05; A61K 31/715; A61P 21/00

(52) U.S. Cl. 514/54; 514/563; 514/736

(57) ABSTRACT

The biological function of skeletal muscle precursor cells in the repair and growth of skeletal muscle in response to exercise is promoted by providing a supplemental composition comprising at least creatine and fucoidin to reinforce biochemical pathways involved in the maintenance of skeletal muscle satellite cells and other myogenic precursors. The composition and method of the present invention induce muscle hypertrophy via satellite cells fusion to muscle fibres and induce a substantially simultaneous replenishment of myogenic precursor cells in response to exercise in a mammal.

### FIELD OF THE INVENTION

The present invention is directed towards a composition and method for inducing muscle hypertrophy via satellite cells fusion to muscle fibres and for inducing a substantially simultaneous replenishment of myogenic precursor cells in response to exercise.

### BACKGROUND OF THE INVENTION

Body composition, including muscle, is influenced both by genetic factors and environmental stimuli. Important environmental factors or stimuli which effect muscle metabolism include food intake and exercise (Rennie M J. Body maintenance and repair: how food and exercise keep the musculoskeletal system in good shape. Exp Physiol. July 2005;90(4):427-36). Gene and protein expression patterns change in response to stimuli. This results in muscle adaptations such as muscle atrophy (loss) or muscle hypertrophy (gain). The determination of muscle loss or gain is the net effect of both positive and negative factors governing muscle development.

‘True’ muscle hypertrophy can be defined as “as an increase in fiber diameter without an apparent increase in the number of muscle fibers, accompanied by enhanced protein synthesis and augmented contractile force” (Sartorelli V, Fulco M. Molecular and cellular determinants of skeletal muscle atrophy and hypertrophy. Sci STKE. Jul. 27, 2004;2004(244):re 11). However, postnatal muscle growth is known to involve both myofiber hypertrophy and increased numbers of myonuclei—the source of which are satellite cells (Olsen S, Aagaard P, Kadi F, Tufekovic G; Verney J, Olesen J L, Suetta C, Kjaer M. Creatine supplementation augments the increase in satellite cell and myonuclei number in human skeletal muscle induced by strength training. J Physiol. Jun. 1, 2006;573(Pt 2):525-34). The growth in the size of muscles after birth is, in reality, a combination of an increase in the actual diameter of a given muscle fiber and an increase in the number of mononuclei.

Satellite cells are a small population of quiescent muscle precursor cells that occupy a “satellite” position immediately outside of muscle fibers (Mauro A. Satellite cell of skeletal muscle fibers. J Biophys Biochem Cytol. February 1961;9:493-5). They are normally maintained in a quiescent state and become activated to fulfill roles of routine maintenance, repair and hypertrophy. Satellite cells are thought to be muscle-specific stem cells which are capable of producing large numbers of differentiated progeny as well as being capable of self-renewal (Collins C A, Partridge T A. Self-renewal of the adult skeletal muscle satellite cell. Cell Cycle. October 2005;4(10):1338-41). In order that satellite cells can fulfill their biological role, they must become activated, proliferate, differentiate and fuse to existing muscle cells (Anderson J E. The satellite cell as a companion in skeletal muscle plasticity: currency, conveyance, clue, connector and colander. J Exp Biol. June 2006;209(Pt 12):2276-92). In this way, multinucleate muscle fibers are maintained or increased in size in response to stimuli.

Activation of satellite cells is essential for their proper function and is defined as “an entry into G1 from quiescence and mobilization” (Anderson J E, Wozniak A C. Satellite cell activation on fibers: modeling events in vivo—an invited review. Can J Physiol Pharmacol. May 2004;82(5):300-10). One of the main factors which has been associated with the activation of satellite cells is nitric oxide (NO) (Anderson J E, Wozniak A C. Satellite cell activation on fibers: modeling events in vivo—an invited review. Can J Physiol Pharmacol. May 2004;82(5):300-10). NO is a small, freely diffusible signaling molecule produced in muscle by neuronal NO-synthase. NO release is regulated by stretching in skeletal muscle and is thought to be responsible for early satellite cell activation in response to muscle injury in proximal and distal muscle fibers (Anderson J E. A role for nitric oxide in muscle repair: nitric oxide-mediated activation of muscle satellite cells. Mol Biol Cell. May 2000;11(5):1859-74).

NO activity is largely controlled by regulating the enzymes responsible for synthesizing NO—Nitric Oxide Synthases (NOSs). All major nitric oxide synthase (NOS) isoforms and splice variants, including a muscle-specific splice variant, are expressed in the skeletal muscles of all mammals (Stamler J S, Meissner G. Physiology of nitric oxide in skeletal muscle. Physiol Rev. January 2001;81(1):209-237). Furthermore, the inner lining, or endothelium, of blood vessels uses NO to signal the surrounding smooth muscle to relax. This has the effect of dilating the artery thereby increasing blood flow in the affected region.

NO has also been shown to play an important role in myoblast and satellite cell fusion (Pisconti A, Brunelli S, Di Padova M, De Palma C, Deponti D, Baesso S, Sartorelli V, Cossu G, Clementi E. Follistatin induction by nitric oxide through cyclic GMP: a tightly regulated signaling pathway that controls myoblast fusion. J Cell Biol. Jan. 16, 2006;172(2):233-44) thereby contributing to muscle maintenance and growth. Myoblast fusion is itself a complex process involving migration, recognition and adhesion, each involving several mechanisms and factors.

If stem cells are to function properly, their pools must be maintained. Therefore, a defining feature of stem cells is their ability of self-renewal in addition to being able to produce differentiated cells (Collins C A, Partridge T A. Self-renewal of the adult skeletal muscle satellite cell. Cell Cycle. October 2004;4 (10):1338-41). Research has shown that the pool of satellite cells is maintained not only by self-renewal but also by contributions from the hematopoietic, i.e. blood, system (Doyonnas R, LaBarge M A, Sacco A, Chariton C, Blau H M. Hematopoietic contribution to skeletal muscle regeneration by myelomonocytic precursors. Proc Natl Acad Sci USA. Sep. 14, 2004;101(37):13507-12).

In the case where hematopoietic stem cells contribute to muscle maintenance, the cells must migrate to the area at which they are required for repair or maintenance. This directed migration of stem/progenitor cells is termed ‘mobilization’. A main mechanism for the mobilization of stem cells is through the release of signaling molecules at the site of the stem cell requirement wherein the stem cells express the corresponding cell surface receptors (Papayannopoulou T. Current mechanistic scenarios in hematopoietic stem/progenitor cell mobilization. Blood. Mar. 1, 2004;103(5):1580-5). An important receptor-ligand system for the relationship between the hematopoietic and muscle systems is the CXCR-4 receptor and the secreted chemokine, SDF-1 (Ratajczak M Z, Majka M, Kucia M, Drukala J, Pietrzkowski Z, Peiper S, Janowska-Wieczorek A Expression of functional CXCR4 by muscle satellite cells and secretion of SDF-1 by muscle-derived fibroblasts is associated with the presence of both muscle progenitors in bone marrow and hematopoietic stem/progenitor cells in muscles. Stem Cells. 2003;21 (3):363-71).

The foregoing disclosure describes a composition and method for stimulating the aforementioned for purposes of muscle hypertrophy in a mammal.

### SUMMARY OF THE INVENTION

The present invention comprises, in accordance with an embodiment therof, the administration, to a mammal, of a composition comprising at least creatine or pharmaceutically acceptable derivatives of creatine such as salts and esters of creatine and a source of fucoidan, to induce muscle hypertrophy via satellite cell fusion to muscle fibres and to provide substantially coincident support for the replenishment of myogenic precursor cells in response to exercise.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards inducing muscle hypertrophy via satellite cell fusion to muscle fibres and provide substantially coincident support for the replenishment of myogenic precursor cells in response to exercise. The composition and method of the present invention accomplishes said support by encouraging multiple distinct aspects of muscle-specific progenitor cell biology.

As used herein, the term “muscle-specific progenitor cell” refers to any undifferentiated cell that is, or will be, at any time, capable of differentiating into any cell type which contributes to mature, functional adult skeletal muscle. This, for the purposes of the present disclosure, includes satellite cells and any other multi-potent cells such as hematopoietic stem cells which have the potential to contribute to skeletal muscle hypertrophy and growth, development or maintenance. It is herein understood that, despite a lack of consensus regarding nomenclature, there exists a continuum of cell types that lie between a classical pluripotent ‘embryonic stem cell’ capable of giving rise to all cell types on one extreme and a terminally-differentiated cell on the other extreme. It is herein understood that a number of undifferentiated cell types having increasingly limited developmental potential progressing from an embryonic stem cell toward a terminally-differentiated cell exist between said extremes. Furthermore, it is herein understood that such undifferentiated cells with limited, yet still multiple, developmental possibilities are generally termed ‘tissue-specific stem cells’. However, undifferentiated cells with only one developmental possibility are generally termed ‘progenitor cells’.

Ingredients of the present composition may also be fine-milled in order to improve the immediacy of absorption, and thus the rate of bioavailability upon consumption by an individual. The fine-milling techniques and the immediacy of absorption employed in the present invention are disclosed in U.S. patent application Ser. No. 11/709,526, entitled “Method For Increasing The Rate And Consistency Of Bioavailability Of Supplemental Dietary Ingredients” and U.S. patent application Ser. No. 11/709,525, entitled “Method for a Supplemental Dietary Composition Having a Multi-Phase Dissolution Profile,” both herein incorporated fully by reference. Briefly, rate of bioavailability is increased via a narrowing of particle size range and a concomitant reduction in the average particle size, improving the immediacy of absorption of said supplemental dietary ingredient. Furthermore, the consistency of dissolution, and thus the absorption of orally administered supplemental dietary ingredients, is improved by the fine-milled process.

As used herein, the term “fine-milled” and/or “fine-milling” refers to the process of micronization. Micronization is a mechanical process that involves the application of force to a particle, thereby resulting in a reduction in the size of the particle. The force, in the case of micronization may be applied in any manner such as, e.g., the collision of particles at high rates of speed, grinding, or by an air-jet micronizer. In a preferred embodiment, fine-milled particles are obtained by jet-milling with nitrogen and compressed air.

As used herein, the term “particle size” refers to the diameter of the particle. The term “average particle size” means that at least 50% of the particles in a sample will have the specified particle size. Preferably, at least 80% of the particles in a sample will have the specified particle size, and more preferably, at least 90% of the particles in a given sample will have the specified particle size. For the purposes of the present invention, the preferred particle size range for fine-milled particles is between 2 and 50 microns.

The size of a particle can be determined by any of the methods known within the art. Methods for particle size determination which may be employed are, for example, sieves, sedimentation, electrozone sensing (Coulter counter), microscopy, and/or Low Angle Laser Light Scattering. The preferred methods for the particle size determination of the present invention are the methods which are most commonly used in the pharmaceutical industry, such as laser diffraction, e.g., via light scattering Coulter Delsa 440SX.

The fine-milling process may be employed in the processing of one or more of the ingredients of the present invention in the dosage forms of tablets, e.g., immediate-release film coated, modified-release and fast-dissolving; capsules, e.g., immediate-release and modified-release; liquid dispersions; powders; drink mixes, etc.

Creatine

Creatine use has been thoroughly studied and is well-established as a beneficial dietary supplement for replenishing energy stores in working muscle cells (Greenhaff P L, Bodin K, Soderlund K, Hultman E. Effect of oral creatine supplementation on skeletal muscle phosphocreatine resynthesis. Am J Physiol. May 1994;266(5 Pt 1):E725-30). The resultant increase in muscular energy stores from creatine supplementation in an individual, combined with physical exercise leads to increased strength, and a reduction in fatigue resulting from high-intensity exercise (Greenhaff P L, Casey A, Short A H, Harris R, Soderlund K, Hultman E. Influence of oral creatine supplementation of muscle torque during repeated bouts of maximal voluntary exercise in man. Clin Sci (Lond). May 1993;84(5):565-71) as well as increasing muscle growth (Volek J S, Duncan N D, Mazzetti S A, Staron R S, Putukian M, Gomez A L, Pearson D R, Fink W J, Kraemer W J. Performance and muscle fiber adaptations to creatine supplementation and heavy resistance training. Med Sci Sports Exerc. August 1999;31(8):1147-56).

More recently however, creatine supplementation has been shown to augment the increase in satellite cell numbers in response to exercise (Olsen S, Aagaard P, Kadi F, Tufekovic G, Verney J, Olesen J L, Suetta C, Kjaer M. Creatine supplementation augments the increase in satellite cell and myonuclei number in human skeletal muscle induced by strength training. J Physiol. Jun. 1, 2006;573(Pt 2):525-34). Furthermore, it was suggested by the results of Olsen et al., that the rate of fusion of satellite cells was also increased in response to creatine supplementation and exercise, adding to an observed increase in the size of muscle fibers.

In various embodiments of the present invention, which are set forth in greater detail in Examples 1 to 4 below, the supplemental composition comprises creatine or derivatives thereof. A serving of the supplemental composition comprises from about 1.00 g to about 10.00 g of creatine or derivatives thereof. The preferred dosage of a serving of the supplemental composition of the present invention comprises about 3.50 g of creatine or pharmaceutically acceptable derivatives of creatine such as salts and esters of creatine.

For example, the creatine may be present in various embodiments of the present invention as creatine salts of malate, maleate, fumarate, tartrate, citrate, succinate, pyruvate, pyroglutamate, glutamate or any other pharmaceutically acceptable salt as known in the art.

Additionally or alternatively, the creatine may be present in various embodiments of the present invention as creatine esters of phosphate, sulphate or any other pharmaceutically acceptable esters as known in the art.

The present invention, as set forth in greater detail in Example 4 below, may further comprise creatine pyroglutamate as a pharmaceutically acceptable derivative of creatine. A serving of the supplemental composition may comprise from about 0.005 g to about 0.10 g of creatine pryoglutamate. The preferred dosage of a serving of the supplemental composition comprises about 0.01 g of creatine pyroglutamate.

The present invention may further comprise at least a portion of the creatine or pharmaceutically acceptable salts or esters thereof in a fine-milled format. In various embodiments of the present invention, the supplemental composition comprises fine-milled creatine or pharmaceutically acceptable salts or esters of said creatine. A serving of the supplemental composition comprises from about 0.005 g to about 0.05 g of fine-milled creatine or pharmaceutically acceptable salts or esters of said creatine. The preferred dosage of a serving of the supplemental composition comprises about 0.02 g of fine-milled creatine or pharmaceutically acceptable salts or esters of said creatine.

Fucoidan

Fucoidans are naturally-occurring sulfated sugar polymers. They are constituents of edible seaweed and have been consumed by humans for centuries. The specific type of fucoidan differs dependent upon the source. Brown seaweed, in particular, is a source of branched-chain Fucoidans and several species have been used experimentally as a source of Fucoidans including Fucus vesiculosus, Undaria pinnatifida and Laminaria japonica. One of the main and earliest mechanisms elucidated for the activity of Fucoidans has been the binding with L- and P-selectin, members of a family of cell surface receptors involved in the inflammatory response. The selectins mediate the binding and adhesion of cells expressing the selectins to other cells such as those on the endothelium upon cytokine activation (Bevilacqua M P, Nelson R M. Selectins. J Clin Invest. February 1993;91(2):379-87).

A number of potential beneficial uses have been suggested for Fucoidan (Berteau O, Mulloy B. Sulfated fucans, fresh perspectives: structures, functions, and biological properties of sulfated fucans and an overview of enzymes active toward this class of polysaccharide. Glycobiology. June 2003;13(6):29R-40R). Fucoidan has been shown to have immunomodulating effects by stimulating lymphocytes and macrophages (Choi E M, Kim A J, Kim Y O, Hwang J K. Immunomodulating activity of arabinogalactan and fucoidan in vitro. J Med Food. 2005 Winter;8(4):446-53). As well, Herpes virus reactivation can be inhibited by treatment with Fucoidan (Cooper R, Dragar C, Elliot K, Fitton J H, Godwin J, Thompson K. G F S, a preparation of Tasmanian Undaria pinnatifida is associated with healing and inhibition of reactivation of Herpes. BMC Complement Altern Med. Nov. 20, 2002;2:11). Importantly, orally administered Fucoidan in humans has been shown to increase the number of CXCR-4-expressing stem cells which can replenish the pool of satellite cells (Irhimeh M R et al. Fucoidan and CXCR4+ hemopoietic progenitor stem cell population, 2004, The Sydney Convention Centre North, Darling Harbour, Australian StemCell Centre). Furthermore, fucoidan can induce the mobilization of these stem cells to muscles (Sweeney E A, Priestley G V, Nakamoto B, Collins R G, Beaudet A L, Papayannopoulou T. Mobilization of stem/progenitor cells by sulfated polysaccharides does not require selectin presence. Proc Natl Acad Sci USA. Jun. 6, 2000;97(12):6544-9). This effect is most likely due to the observed effect of increasing SDF-1 plasma levels (Sweeney E A, Lortat-Jacob H, Priestley G V, Nakamoto B, Papayannopoulou T. Sulfated polysaccharides increase plasma levels of SDF-1 in monkeys and mice: involvement in mobilization of stem/progenitor cells. Blood. Jan. 1, 2002;99(1):44-51).

In a preferred embodiment of the present invention, the supplemental composition comprises fucoidan. A serving of the supplemental composition comprises from about 0.01 g to about 0.1 g of fucoidan. The preferred dosage of a serving of the supplemental composition of the present invention comprises about 0.024 g of fucoidan.

In various embodiments of the present invention, which are set forth in greater detail in Examples 1 to 4 below, the supplemental composition comprises Laminaria japonica extract as a source of fucoidan.

Sphinoglipids

Sphingolipids are important constituents of eukaryotic organisms. Complex sphingolipids and their metabolic products are highly bioactive molecules which are involved in the regulation of many important biological functions including cell growth, differentiation and apoptosis (Vesper H, Schmelz E M, Nikolova-Karakashian M N, Dillehay D L, Lynch D V, Merrill A H Jr. Sphingolipids in food and the emerging importance of sphingolipids to nutrition. J Nutr. July 1999;129(7):1239-50). Sphingolipid metabolism and biological function can be modulated by dietary intake of sphingolipids. Supplying supplemental sphingolipids promotes the synthesis of sphingosine 1-phosphate.

Sphingosine 1-phosphate is a bioactive sphingolipid metabolite that has been shown to regulate a number of important biological functions including satellite cell activation (Nagata Y, Partridge T A, Matsuda R, Zammit P S. Entry of muscle satellite cells into the cell cycle requires sphingolipid signaling. J Cell Biol. Jul. 17, 2006;174(2):245-53) and myogenic differentiation (Donati C, Meacci E, Nuti F, Becciolini L, Farnararo M, Bruni P. Sphingosine 1-phosphate regulates myogenic differentiation: a major role for S1P2 receptor. FASEB J. March 2005;19(3):449-51), both of which are processes important for increasing muscle hypertrophy.

In embodiments of the present invention, which are set forth in greater detail in Examples 2 to 4 below, the supplemental composition comprises a source of sphingolipids. A serving of the supplemental composition of the present invention comprises from about 0.005 g to about 0.05 g of a source of sphingolipids. The preferred dosage of a serving of the supplemental composition of the present invention comprises about 0.014 g of a source of sphingolipids.

Resveratrol

Resveratrol is a polyphenol found in many plant sources, most notably in grape skins, grape juice and red wine. One of the most abundant sources is from the roots of Polygonum cuspidatum. In plants, the biological function of resveratrol is as an antibiotic to fight infection. However, as a component of the diet, either as a constituent of plant-based foods or as a nutritional supplement, resveratrol has been reported to confer many health benefits. The main beneficial function of polyphenols from plant sources is generally attributed to antioxidant activity. However, resveratrol has been shown to increase NO production by tissue-specific induction of NOSs (Das S, Alagappan V K, Bagchi D, Sharma H S, Maulik N, Das D K. Coordinated induction of iNOS-VEGF-KDR-eNOS after resveratrol consumption: a potential mechanism for resveratrol preconditioning of the heart. Vascul Pharmacol. April-May 2005;42(5-6):281-9 Abstract).

In various embodiments of the present invention, which are set forth in greater detail in Examples 3 and 4 below, the supplemental composition of the present invention comprises Polygonum cuspidatum as a source of resveratrol. A serving of the supplemental composition of the present invention comprises from about 0.001 g to about 0.01 g of Polygonum cuspidatum as a source of resveratrol. The preferred dosage of a serving of the supplemental composition of the present invention comprises about 0.004 g of Polygonum cuspidatum as a source of resveratrol.

In a preferred embodiment of the present invention, the composition of the present invention comprises at least creatine or pharmaceutically acceptable derivatives of creatine such as salts and esters of creatine and a source of fucoidan.

In another embodiment of the present invention, the composition of the present invention comprises creatine or pharmaceutically acceptable derivatives of creatine such as salts and esters of creatine, a source of fucoidan and a source of sphingolipids.

In yet another embodiment of the present invention, the composition of the present invention comprises creatine or pharmaceutically acceptable derivatives of creatine such as salts and esters of creatine, a source of fucoidan, a source of sphinoglipids and a source of resveratrol.

The compositions of the present invention may also comprise, in addition to the aforementioned constituents, any number of amino acids in sufficient quantities to be effective in inducing muscle hypertrophy, or salts or esters of said amino acids. For example, proteins, such as whey protein, casein protein, milk proteins, or soy protein, may further be included in the compositions of the present invention in quantities effective to induce muscle hypertrophy. Amino acids and proteins are well known in the art to aid in the generation and repair of muscle protein.

Not wishing to be bound by theory, it is believed that the various components of the present invention will act via multiple, distinct biological pathways to aid in the maintenance and function of myogenic precursor cells in repair and growth of skeletal muscle. The composition of the present invention, when used in conjunction with the method provided herein, induces muscle hypertrophy via satellite cells fusion to muscle fibres and induces the substantially simultaneous replenishment of myogenic precursor cell in response to exercise.

Additionally, by way of ingestion of the composition of the present invention, a method for enhancing the effectiveness of the immune system in an individual is provided. The method of the present invention comprises at least the step of administering to an individual a therapeutically effective and acceptable amount of the composition of the present invention.

According to additional methods, the compositions of the present invention may be administered to a mammal via any therapeutically acceptable format. For example, the compositions of the present invention may be administered to a mammal intravenously, intramuscularly, or interperitoneally as routes of administration distinct from the aforementioned oral method. These instantly disclosed routes of administration may also be combined with an oral administration of the composition of the present invention as an additional method of administration to a mammal.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule. The present composition may also be provided in various time-release formats, e.g. a slow-release format, a quick-release format, or a phase-release format, as are known in the art as well.

Furthermore, the dosage form of the nutritional supplement may be provided in accordance with customary processing techniques for herbal and nutritional supplements in any of the forms mentioned above. Additionally, the nutritional supplement set forth in the example embodiments herein may contain any appropriate number and type of excipients, as is well known in the art.

The present nutritional composition or those similarly envisioned by one of skill in the art, may be utilized in methods to support the biological function of muscle-specific progenitor cells required for skeletal muscle recovery and growth in response to exercise. Specifically, the present compositions and method disclosed herein are provided to induce muscle hypertrophy via satellite cell fusion to muscle fibres and induce a substantially coincident replenishment of myogenic precursor cell in response to exercise.

Although the following examples illustrates the practice of the present invention in several of its embodiments, the examples should not be construed as limiting the scope of the invention. Other embodiments will be apparent to one of skill in the art from consideration of the specifications and examples.

### EXAMPLES

### Example 1

A nutritional supplement for inducing muscle hypertrophy via satellite cell fusion to muscle fibres and inducing a substantially simultaneous replenishment of myogenic precursor cell in response to exercise is provided. The nutritional supplement is in the form of caplets. One serving of the nutritional supplement is 7 caplets and each serving comprises:
about 3.5 g of creatine monohydrate (fine-milled), and about 0.024 g of Laminaria japonica extract (standardized to 85% fucoidan).

### Example 2

A nutritional supplement for inducing muscle hypertrophy via satellite cells fusion to muscle fibres and inducing a substantially simultaneous replenishment of myogenic precursor cell in response to exercise is provided. The nutritional supplement is in the form of caplets. One serving of the nutritional supplement is 7 caplets and each serving comprises:
about 3.5 g of creatine monohydrate (fine-milled), about 0.024 g of Laminaria japonica extract (standardized to 85% fucoidan), and about 0.0145 g of soy/milk sphingolipids.

### Example 3

A nutritional supplement for inducing muscle hypertrophy via satellite cells fusion to muscle fibres and inducing a substantially simultaneous replenishment of myogenic precursor cell in response to exercise is provided. The nutritional supplement is in the form of caplets. One serving of the nutritional supplement is 7 caplets and each serving comprises:
about 3.5 g of creatine monohydrate (fine-milled), about 0.024 g of Laminaria japonica extract (standardized to 85% fucoidan), about 0.0145 g of soy/milk sphingolipids, and about 0.004 g of Polygonum cuspidatum.

### Example 4

A nutritional supplement for inducing muscle hypertrophy via satellite cells fusion to muscle fibres and inducing a substantially simultaneous replenishment of myogenic precursor cell in response to exercise is provided. The nutritional supplement is in the form of caplets. One serving of the nutritional supplement is 7 caplets and each serving comprises:
about 2.00 g of creatine monohydrate (fine-milled), about 0.02 g of creatine monohydrate (nanodiffuse), about 0.01 g of creatine pyroglutamate, about 1.30 g of creatine citrate, about 0.001 g of creatine malate, about 0.02 g of creatine pyruvate, about 0.024 g of Laminaria japonica extract (standardized to 85% fucoidan), about 0.0145 g of soy/milk sphingolipids, about 0.004 g of Polygonum cuspidatum, about 0.05 g of L-glycine ethyl ester, about 2.9 g of L-tyrosine, about 0.001 g of L-tyrosine methyl ester, and about 0.002 g of Yohimbe.

Extensions and Alternatives

In the foregoing specification, the invention has been described with specific embodiments thereof. However, it will be evident that various modifications and changes may be made thereto without departing from the broader spirit and scope of the invention.

What is claimed:

1. A composition comprising creatine or derivatives thereof and a source of fucoidan in amounts effective to induce muscle hypertrophy and to induce a substantially simultaneous replenishment of myogenic precursor cells in a mammal.

2. The composition of claim 1, wherein muscle hypertrophy is induced via satellite cells fusion to muscle fibres in a mammal.

3. The composition of claim 1, further comprising a source of sphingolipids.

4. The composition of claim 3, wherein the source of sphingolipids is provided in an amount effective to promote the synthesis of sphingosine 1-phosphate in a mammal.

5. The composition of claim 3, further comprising a source of resveratrol.

6. The composition of claim 5, wherein the source of resveratrol is provided in an amount effective to increase the production of nitric oxide in a mammal.

7. A method comprising at least the step of administering to a mammal a composition comprising creatine or derivative thereof and a source of fucoidan wherein said composition induces muscle hypertrophy and substantially simultaneously induces a replenishment of myogenic precursor cells in said mammal in response to physical exercise.

8. The method of claim 7, wherein the composition further comprises a source of sphingolipids.

9. The method of claim 8, wherein the source of sphingolipids promote the synthesis of sphingosine 1-phosphate in the mammal.

10. The method of claim 7, wherein the composition further comprises a source of resveratrol.

11. The method of claim 10, wherein the source of resveratrol increases the production of nitric oxide in the mammal.
